SEQUENCE LISTING (6 sequences)

SEQ ID NO: 1 - DNA, 31 bp, Unknown
  Forward PCR primer used to amplify CP fragment
acgtctagaaaccacgtcgactccactccat

SEQ ID NO: 2 - DNA, 29 bp, Unknown
  Reverse PCR primer used to amplify CP fragment
agcatcgatagcttgccgctcaccagcac

SEQ ID NO: 3 - DNA, 34 bp, Unknown
  Forward PCR primer used to amplify R2 fragment
acgtctagatttagcaaaacgcagcacaaaatca

SEQ ID NO: 4 - DNA, 32 bp, Unknown
  Reverse PCR primer used to amplify R2 fragment
agcatcgattctccagacaccatgattgggtg

SEQ ID NO: 5 - DNA, 33 bp, Unknown
  Forward PCR primer used to amplify R3 fragment
acgtctagattcgctttaaaacagaaagcacca

SEQ ID NO: 6 - DNA, 30 bp, Unknown
  Reverse PCR primer used to amplify R3 fragment
agcatcgatgcctcgtagcttggttgggtt

(19) United States
(12) Patent Application Publication
(10) Pub. No.: US 2010/0281568 A1
(43) Pub. Date: Nov. 4, 2010

(54) TRANSFORMATION OF POINSETTIA PLANTS

(76) Inventors: Jihong Liu Clarke, Moss (NO); Dag Ragnar Blystad, Aas (NO); Sissel Britt Haugslien, Spydeberg (NO)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 12/678,328

(22) PCT Filed: Sep. 17, 2008
(86) PCT No.: PCT/GB08/03147

§ 371 Date: Jun. 28, 2010

(30) Foreign Application Priority Data
Sep. 17, 2007 (GB) 0718112.6

(51) Int. Cl. A01H 5/00; C12N 15/87; C12N 15/82

(52) U.S. Cl. 800/279; 800/294; 800/323

(57) ABSTRACT

The invention relates to a process for the Agrobacterium-mediated transformation of Euphorbia pulcherrima (Poinsettia). The invention also relates to Euphorbia pulcherrima plants which have been transformed in this way and to plant material obtained therefrom.

The invention relates to a process for the Agrobacterium-mediated transformation of Euphorbia pulcherrima (Poinsettia). The invention also relates to Euphorbia pulcherrima plants which have been transformed in this way and to plant material obtained therefrom.

Poinsettia (Euphorbia pulcherrima Willd. Ex Klotzsch) is a contemporary symbol of Christmas in most parts of the world. Consumption of flowers in Norway continues to increase 5-10% per year. Ornamentals produced in greenhouses have a value of approximately 40% of the total first-hand value of the production in horticulture in Norway (Statistics 2003-2004 from the Norwegian Grower's Association (NGF)). In Norway, poinsettia is one of the most important pot plants with a yearly production close to 6 million plants. The annual production in the USA and the EU is 50 million and 100 million plants, respectively. Today, Europe and North America represent the largest volume of production and sales, but demand is growing quickly in Australasia as poinsettia becomes more popular each year (Williams 2005). Its ornamental value and innovation potential has laid the basis for extensive research in Norway.

For many years, the focus from both the grower's association (G3 Ungplanter) and academia has been on creating new cultivars with improved ornamental values such as enhanced quality and resistance to diseases in existing poinsettia. Traditionally, the improvement of quality and ornamental values for poinsettia has been achieved by conventional breeding methods. Breeders have managed to provide new varieties in flower colour, vigour, leaf form, and pest and disease resistance etc. using known methods. However, traditional breeding is limited to variations which exist in the gene pools of the parent species; these are not enough to meet the markets' demands. Sexual incompatibility is another factor restricting the utilization of genetic resources by traditional breeding approaches. With advances in technology, a number of new techniques, such as in vitro breeding techniques, embryo rescue, in vitro pollination, protoplast fusion and mutagenesis have been developed to complement the weaknesses of conventional breeding. However, another common disadvantage of both traditional and in vitro breeding techniques is the long, time-consuming backcrossing process. This can often take many years and has limited the number of commercially-viable cultivars to date.

Genetic transformation technology can overcome the limitations of the gene pool, sexual barriers and the necessity for subsequent backcrossing. Genetic transformation has been utilized in crop improvement conferring important agronomic traits including disease resistance against various pathogens, in addition to many other important traits. This technology overcomes the difficulties encountered by traditional approaches and can directly introduce desirable genes/agronomic traits into a target plant efficiently.

Viral diseases in plants can cause serious economic losses every year. Unlike fungal diseases where chemical method can be used to successfully prevent diseases, control of plant virus diseases has been more problematic. Control strategies have consisted mainly of approaches aimed at reducing or eliminating existing sources of infection within or outside the target crop, prevention of virus transmission, or minimizing the effects of infection. Cross protection and breeding for resistant or tolerant cultivars have been the main approaches applied worldwide. For ornamental plants, genetic transformation has been utilized in many flowers with desirable horticultural properties and enhanced ornamental values.

Poinsettia Mosaic Virus (PnMV) is a disease which causes damage to poinsettia cultivars. PnMV causes visible symptoms in poinsettia during parts of the growing season. These symptoms consist of light green areas—mosaics—in the leaves (FIG. 1). This mosaicing is most pronounced in the green leaves on the border to the coloured top leaves (FIG. 1).

Therefore there is considerable interest in the potential benefits of growing PnMV-free poinsettias. Traditionally, PnMV-free poinsettia plants were obtained by in vitro culture of apical meristems. However, it is a time-consuming and labour intensive method.

Conditions for the transformation of the cells of many plants with Agrobacterium are well known in the art. However, conditions which are suitable for the transformation of Euphorbia cells have not previously been known. In light of this, biolistic approaches for the transformation of Poinsettia have been developed (e.g. U.S. Pat. No. 7,119,262). However, this has several disadvantages, e.g. random integration of the transgene, multiple copies of the transgene, gene silencing, instability of transgene etc.

To overcome the difficulties encountered by traditional approaches and some of the disadvantages of the biolistic method, an Agrobacterium-mediated transformation method for Euphorbia pulcherrima has at last been developed. The methodology developed facilitates the improvement of ornamental Euphorbia pulcherrima plants with aims to enhance their disease resistance, quality traits and ornamental values.

The invention therefore relates to a process for producing a transformed Euphorbia pulcherrima plant, comprising transforming Euphorbia pulcherrima plant material with Agrobacterium containing a gene construct and regenerating a transformed Euphorbia pulcherrima plant from the transformed plant material.

In one embodiment, the invention provides a process for producing a transgenic Euphorbia pulcherrima somatic embryo, comprising the steps of:

(a) culturing a transformable Euphorbia pulcherrima plant material, wherein the plant material is a plant cell, plant tissue, explant or protoplast, on or in a callus inducing media comprising CPA and BAP;
(b) inoculating the transformable Euphorbia pulcherrima plant material with Agrobacterium which contains a gene construct comprising a selectable marker gene and a reporter gene and which is capable of transferring the gene construct to the plant material;
(c) culturing the transformable Euphorbia pulcherrima plant material after inoculation with Agrobacterium on or in a callus inducing media comprising CPA and BAP in the absence of the selection agent and an anti-Agrobacterium agent;
(d) culturing the transformable Euphorbia pulcherrima plant material on or in a callus inducing media comprising CPA and BAP in the presence of an anti-Agrobacterium agent and the selection agent;
(e) culturing the embyrogenic calli thus produced on or in a somatic embryo induction media comprising NAA and 2iP in the presence of an anti-Agrobacterium agent and the selection agent;
(f) culturing the somatic embryos thus produced on or in a somatic embryo induction media comprising NAA and 2iP in the presence of an anti-Agrobacterium agent and the selection agent, and optionally subculturing the somatic embryos one or more additional times.

In some embodiments, the process additionally includes the step:

(g) culturing the somatic embryos on or in a somatic embryo maturation media comprising BAP.

In other embodiments, the process includes the additional steps:

(g) culturing the somatic embryos on or in a somatic embryo maturation media comprising BAP; and
(h) culturing plantlets derived from the somatic embryos on or in root induction media.

Preferably, the Euphorbia is Euphorbia pulcherrima Willd. Ex Klotzsch (poinsettia).

A particularly preferred Euphorbia pulcherrima is Euphorbia pulcherrima (cv. Millennium). Other examples of Euphorbia pulcherrima cultivars include Cristella, Angelika, Lilo and Cortez.

In the context of the present invention, the term “transformable Euphorbia pulcherrima plant material” refers to a Euphorbia pulcherrima plant cell, plant tissue, explant or plant protoplast which is capable of being transformed with a genetic construct, such as a Ti-DNA or derivative thereof, from an Agrobacterium species.

Examples of plant cells, plant tissues, explants or plant protoplasts include inter alia leaves, leaf explants, stems, stem explants, shoot apices, roots, hypocotyls, cotyledons, seeds, pollen, calli, immature and somatic embryos, shoot apical meristems, etc.

Preferably, the plant material is a plant cell, callus, immature or somatic embryo, shoot apical meristem, leaf explant, stem explant or protoplast.

Examples of cells include cells obtained from the aforementioned materials and calli derived from various parts of the plant. Examples of explants includes explants obtained from the aforementioned materials.

The plant from which the plant material is obtained is preferably a young plant, more preferably one which is 4-12 weeks old, most preferably one which is 6-8 weeks old.

In some embodiments, the plant material is a stem, leaf, zygotic embryo, somatic embryo, embryogenic calli, apical meristem, or a cell obtained therefrom or suspension of cultured cells or protoplasts.

Preferably, the plant tissue which is transformed is a stem explant, most preferably an internode stem explant, and particularly preferably a stem explant which has been taken from right below the shoot apical meristem. Preferably, the stem explants are 0.5-1.5 cm long.

Prior to use, the plant material may be sterilized in order to limit undesirable bacterial growth. For example, stem explants may be sterilized using alcohol and/or NaOCl, followed by rinsing with water.

In Step (a), prior to transformation with Agrobacterium, the plant material is cultured on or in callus inducing (CI) media comprising CPA and BAP.

The general composition of “callus inducing (CI) media” will be well known to persons skilled in the art. One example of such media is MS basal medium comprising macro and micro components and minerals which are necessary for plant growth. (For medium suitable for poinsettia, reference is: Preil, W. 1994. In vitro culture of poinsettia. In E. Strømme (ed.). The scientific basis of poinsettia production. Agricultural University of Norway.)

However, general CI media have previously not enabled the preparation of transformable Euphorbia pulcherrima plant material. The inventors have found that callus inducing (CI) media comprising CPA and BAP is efficacious in this regard.

Preferably, the CI media comprises 0.1-0.35 mg L⁻¹ CPA, more preferably 0.15-0.25 mg L⁻¹ CPA, and most preferably about 0.2 mg L⁻¹ CPA.

Preferably, the CI media comprises 0.1-0.35 mg L⁻¹ BAP, more preferably 0.15-0.25 mg L⁻¹ BAP, and most preferably about 0.2 mg L⁻¹ BAP.

The time that the plant material may be cultured on or in callus inducing (CI) media comprising CPA and BAP is preferably 20-28 hours, more preferably 22-26 hours, and most preferably about 1 day.

The plant material is preferably cultured on or in callus inducing (CI) media comprising CPA and BAP in the dark or under reduced light conditions.

The Agrobacterium used in the invention is one which contains a gene construct comprising a selectable marker gene and a reporter gene and which is capable of transferring the gene construct to the plant material.

Preferably the Agrobacterium is Agrobacterium tumefaciens.

The skilled person will understand that, in order to facilitate the transfer of the gene construct to the plant material, the vir gene must also be present in the Agrobacterium, either in the gene construct or in a separate plasmid/vector.

Preferably, the Agrobacterium is an Agrobacterium tumefaciens which contains a disarmed Ti plasmid which has only the vir and on region of the Ti plasmid.

Most preferably, the Agrobacterium is Agrobacterium tumefaciens strain LBA 4404. The “gene construct comprising a selectable marker gene and a reporter gene” will generally be a nucleic acid vector or nucleic acid plasmid. The skilled person will be familiar with vectors and plasmids which are based upon or derived from the Ti (tumour inducing) plasmid, a large conjugative plasmid naturally found in Agrobacterium tumefaciens.

Within the Ti plasmid is a segment of DNA known as the T-DNA which is flanked by T-DNA borders. Upon transformation of the Ti plasmid into the plant, the T-DNA is inserted into the genome of the plant. This provides a route for inserting a foreign or heterologous nucleic acid molecules into the genome of the plant.

The gene construct comprises at least a selectable marker gene and a reporter gene. In other embodiments, the reporter gene is a selectable marker gene.

In one embodiment, the gene construct is a plasmid or vector which comprises a selectable marker gene and a reporter gene flanked by at least one T-DNA border. Preferably, the {selectable marker gene and reporter gene} are flanked by two T-DNA borders. Where only one T-DNA border is present, the right-hand border is present.

In certain embodiments, the gene construct comprises:

(i) a left Agrobacterium T-DNA border sequence;
(ii) a selectable marker gene comprising:
a promoter which functions in Euphorbia pulcherrima plant cells
a nucleotide sequence encoding a selectable marker polypeptide
a 3′ non-translated region encoding a terminator sequence
   (iii) a reporter gene comprising:
a promoter which functions in Euphorbia pulcherrima plant cells
a nucleotide sequence of interest
a 3′ non-translated region encoding a terminator sequence
   (iv) a right Agrobacterium T-DNA border sequence.

In other embodiments, the order of the selectable marker gene and the reporter gene are reversed.

The promoter may be any plant promoter which is operable in Euphorbia pulcherrima. The preferred promoter is CaMV 35S promoter.

The selectable marker gene encodes a polypeptide which confers resistance to the selection agent. The selection agent is, for example, an antibiotic suitable for plant cell culture; it might also be a herbicide.

Preferably, the selectable marker gene is neomycin phosphotransferase II (nptII) and the selection agent is kanamycin. Examples of other suitable selectable marker genes/selection agents include hygromycin and the herbicide resistance gene bar. Other selection marker genes include Escherichia coli aspartate kinase or LysC, the green fluorescence protein (gfp) and the luciferase gene (reviewed by Stewart 2001, The utility of green fluorescent protein in transgenic plants. Plant Cell Rep. 20: 376-382, incorporated herein by reference).

The nucleic acid molecule of interest in the reporter gene may comprise one or more of the following, inter alia:
A disease resistance gene or pathogenesis related (PR) protein-encoding gene, for example a gene which encodes polypeptide with an anti-bacterial, anti-fungal, insecticidal or anti-viral properties.
A gene which encodes a polypeptide which modifies a known phenotypic trait or introduces a new phenotypic trait to the plant. Examples of such traits include increased leaf production and increased branching, and tolerance to coldness or dryness.
A gene which encodes a polypeptide which modifies the colour of all or part of the plant or affects the ornamental value of the plant. The skilled person will be aware of the considerable demand for Euphorbia, e.g. Poinsettia, which are yellow, pure white, blue or lilac. For colour or quality improvement, the appropriate gene which is responsible for the target trait is used. For example, for producing yellow-coloured poinsettia, the colour biosynthetic pathway may be altered by expressing two transgenes (AmAS1 and Am4′CGT).
A gene-silencing construct, for example one comprising a region of first DNA and a region of second DNA which is complementary to the first DNA, the first and second DNAs being separated by an intron or spacer. Upon transcription, the gene-silencing construct makes a hairpin loop structure. Gene-silencing constructs may particularly be used to stop and/or reduce infection by generating small interfering RNAs (siRNAs) which are homologous to parts of the genome of one or more viruses.

Further examples of nucleic acid molecules of interest include those encoding antibodies, antibiotics, herbicides, vaccine antigens, enzymes, enzyme inhibitors and designer peptides.

A preferred nucleic acid molecule of interest is one which encodes a fragment of the Poinsettia Mosaic Virus (PnMV) coat protein. Preferably, the nucleic acid molecule of interest encodes a fragment of the Poinsettia Mosaic Virus (PnMV) coat protein in sense and antisense orientations, separated by an intron or spacer. Preferably the intron is a pdk intron. Preferably, the fragment is about 500 nucleotides in length.

A further preferred nucleic acid molecule of interest is one which encodes a fragment of an RNA dependent RNA polymerase. Preferably, the nucleic acid molecule of interest encodes a fragment of an RNA dependent RNA polymerase in sense and antisense orientations, separated by an intron or spacer. Preferably the intron is a pdk intron. Preferably, the fragment is about 500 nucleotides in length.

The gene construct may also contain other elements which enable its handling and reproduction, such as an origin of replication, selection elements, and multiple cloning sites.

Preferably, the gene construct is a pHANNIBAL or pKANNIBAL vector (CSIRO).

Agrobacterium containing the gene construct may be prepared by standard means. In general, the steps will involve the preparation of competent Agrobacterium cells; and the introduction of the gene construct into Agrobacterium, for example by electroporation.

Agrobacteria containing the gene construct may be grown overnight in an appropriate liquid media, e.g. LB media (Sigma), supplemented with an appropriate selection agent.

The Agrobacteria are preferably grown in the liquid media with shaking at 150-250 rpm, more preferably at 175-225 rpm, and most preferably at about 200 rpm.

The Agrobacteria are preferably grown overnight at a temperature of 25-31° C., more preferably at a temperature of 27-29° C., and most preferably at a temperature of about 28° C.

The Agrobacteria are grown to an appropriate OD, preferably to OD₆₀₀=0.5-1.0, more preferably 0.55-0.65, most preferably about 0.6.

The selection agent, preferably an antibiotic, is preferably used at a concentration of 25-75 mg L⁻¹, more preferably at a concentration of 40-60 mg L⁻¹, and most preferably at a concentration of about 50 mg L⁻¹.

Once grown, the Agrobacterium may be separated from the liquid media, for example, by pelleting using centrifugation.

The Agrobacterium are then washed to remove traces of the overnight growth media and then resuspended in a suitable media prior to inoculation of the plant material. Preferably, the Agrobacterium are washed with Murashige & Skoog (MS) basal medium supplemented with about 2% sucrose (MS-2), and resuspended in MS-2.

In Step (b), the transformable Euphorbia pulcherrima plant material is inoculated with Agrobacterium which contains a gene construct comprising a selectable marker gene and a reporter gene. The Agrobacterium is one which is capable of transferring the gene construct to the plant material.

As used herein, the term “inoculating” means infecting a Euphorbia pulcherrima plant material or incubating protoplasts from the Euphorbia pulcherrima plant with Agrobacterium.

This inoculation is carried out under conditions which are suitable for the gene construct contained within the Agrobacterium to be transferred to the plant material.

The Agrobacterium (preferably resuspended in MS-2) are applied to the plant material for a time which allows transfer of the gene construct to occur.

In one embodiment, the Agrobacterium are co-cultivated with the plant material for a time which allows the Agrobacterium to attach to the plant material. Preferably this time is 2-20 minutes, more preferably 5-15 minutes and most preferably about 5 minutes.

In Step (c), the transformable Euphorbia pulcherrima plant material are cultured after inoculation on or in a callus inducing (CI) media comprising CPA and BAP in the absence of the selection agent. Furthermore, the CI media should not contain an anti-Agrobacterium agent. At this time, the Agrobacterium have attached themselves to the plant material and the physical transfer of the gene construct occurs.

Previous general CI media have not enabled the Agrobacterium-mediated transformation of Euphorbia pulcherrima. The inventors have found that callus inducing (CI) media comprising CPA and BAP is efficacious in this regard.

The term “anti-Agrobacterium agent” refers to an agent which kills Agrobacterium or reduces significantly the activity of the Agrobacterium.

The Agrobacterium are preferably co-cultured with the plant material at a temperature of 20-28° C., more preferably 22-26° C. and most preferably at about 24° C. Preferably, they are co-cultured for 24-120 hours, more preferably for 48-96 hours, and most preferably for about 72 hours. It is preferred that this co-culturing is carried out in the dark or under reduced light conditions.

In Step (d), after transformation with Agrobacterium, the plant tissue is cultured on or in CI media comprising CPA and BAP in the presence of an anti-Agrobacterium agent and the selection agent.

Preferably, the anti-Agrobacterium agent is claforan. Preferably, the concentration of the anti-Agrobacterium agent is 300-500 mg L⁻¹, and most preferably a concentration of about 400 mg L⁻¹.

The time that the plant material may be cultured on or in the above-defined callus inducing (CI) media is preferably 2-15 days, more preferably 5-12 hours, and most preferably 7-10 days. After this time, embryogenic calli are produced.

Preferably, the light conditions are 20-26 μE m⁻² s⁻¹, most preferably about 23 μE m⁻² s⁻¹. The photoperiod is preferably 14-18 hours, and most preferably about 16 hours.

In Step (e), the embryogenic calli are cultured on or in a somatic embryo inducing media comprising NAA and 2iP in the presence of an anti-Agrobacterium agent and the selection agent.

The general composition of the “somatic embryo inducing media” which is usable in this regard will be well known to persons skilled in the art. One example of such a media is MS basal medium comprising a given percent sucrose content and appropriate hormones.

However, general somatic embryo inducing media have previously not enabled the induction of somatic embryos from transformed Euphorbia pulcherrima plant material. The inventors have found that somatic embryo inducing media comprising NAA and 2iP in the presence of an anti-Agrobacterium agent and the selection agent is efficacious in this regard.

Preferably, the somatic embryo inducing media comprises 0.1-0.4 mg L⁻¹ NAA, more preferably 0.25-0.35 mg L⁻¹ NAA, and most preferably about 0.3 mg L⁻¹ NAA.

Preferably, the somatic embryo inducting media comprises 0.10-0.30 mg L⁻¹ 2iP, more preferably 0.12-0.20 mg L⁻¹ 2iP, and most preferably about 0.15 mg L⁻¹ 2iP.

Preferably the anti-Agrobacterium agent is claforan. Preferably the concentration of the anti-Agrobacterium agent is 300-500 mg L⁻¹, and most preferably at a concentration of about 400 mg L⁻¹.

The time that the embryogenic calli may be cultured on or in the above-defined somatic embryo induction media is preferably 10-18 days, more preferably 12-16 days, and most preferably about 14 days. After this time, somatic embryogenic calli are produced.

Preferably, the light conditions are 20-26 μE m⁻² s⁻¹, most preferably about 23 μE m⁻² s⁻¹. The photoperiod is preferably 14-18 hours, most preferably about 16 hours.

In Step (f), the embryogenic calli are sub-cultured further on or in a somatic embryo inducing media comprising NAA, 2iP, an anti-Agrobacterium agent and the selection agent.

If the selection marker gene is nptII, the selection agent may be kanamycin. Preferably, the concentration of selection agent used in this Step is 10-40 mg L⁻¹, more preferably 20-30 mg L⁻¹ and most preferably about 25 mg L⁻¹.

The anti-Agrobacterium agent is preferably claforan. Preferably, the concentration of anti-Agrobacterium agent used in this Step is 300-700 mg L⁻¹, more preferably 400-600 mg L⁻¹, and most preferably about 500 mg L⁻¹.

The somatic embryos may then be subcultured, for example a further 2-4 times. The globular stage of the somatic embryos becomes visible on the calli after 2-3 subcultures, i.e. after 4-6 weeks. In the further subcultures, the concentration of selection marker may be reduced. For example, if the selection marker gene is nptII, the selection agent may be kanamycin. Preferably, the concentration of kanamycin is reduced to 3-20 mg L⁻¹, more preferably 5-15 mg L⁻¹, and most preferably about 10 mg L⁻¹.

Preferably, the concentration of anti-Agrobacterium agent (e.g. claforan) is reduced to 200-300 mg L⁻¹, more preferably to 225-275 mg L⁻¹, and most preferably to about 250 mg L⁻¹.

Somatic embryos develop after an average of about 10 weeks. These may be transferred to somatic embryo maturation (SEM) media comprising BAP.

The general composition of some “somatic embryo maturation media” will be well known to persons skilled in the art. One example of such media is MS basal medium and a given percent sucrose content. The inventors have found that somatic embryo maturation (SEM) media comprising BAP is particularly efficacious in this regard.

Preferably, the somatic embryo maturation media comprises 0.03-0.07 mg L⁻¹ BAP, more preferably 0.04-0.06 mg L⁻¹ BAP, and most preferably about 0.05 mg L⁻¹ BAP. Preferably, the light conditions are 20-26 μE m⁻² s⁻¹, most preferably about 23 μE m⁻² s⁻¹.

Plantlet derived from somatic embryos may subsequently be cultured on root induction media. Such media are well known in the art.

Examples of root induction media are ½ strength MS comprising 2 mg L⁻¹ IAA and hormone-free ½ strength MS (HFMS) medium.

Preferably, the light conditions are 20-40 μE m⁻² s⁻¹, most preferably about 30 μE m⁻² s⁻¹. The photoperiod is preferably 14-18 hours, most preferably about 16 hours.

Plants with well-developed roots may then be transferred to soil and optionally grown in greenhouses at about 22° C.

The invention also provides a process for producing a transgenic Euphorbia pulcherrima plant, comprising the steps of:

(a) culturing a transformable Euphorbia pulcherrima plant material, wherein the plant material is a plant cell, plant tissue, explant or protoplast, on or in a callus inducing media comprising CPA and BAP;
(b) inoculating the transformable Euphorbia pulcherrima plant material with Agrobacterium which contains a gene construct comprising a selectable marker gene and a reporter gene and which is capable of transferring the gene construct to the plant material;
(c) culturing the transformable Euphorbia pulcherrima plant material after inoculation with Agrobacterium on or in a callus inducing media comprising CPA and BAP in the absence of the selection agent and an anti-Agrobacterium agent;
(d) culturing the transformable Euphorbia pulcherrima plant material on or in a callus inducing media comprising CPA and BAP in the presence of an anti-Agrobacterium agent and the selection agent;
(e) culturing the embyrogenic calli thus produced on or in a somatic embryo induction media comprising NAA and 2iP in the presence of an anti-Agrobacterium agent and the selection agent;
(f) culturing the somatic embryos thus produced on or in a somatic embryo induction media comprising NAA and 2iP in the presence of an anti-Agrobacterium agent and the selection agent, and optionally subculturing the somatic embryos one or more additional times;
(g) culturing the somatic embryos on or in a somatic embryo maturation media comprising BAP; and
(h) culturing plantlets derived from the somatic embryos on or in root induction media in order to produce a transgenic Euphorbia plant.

The invention also provides a Euphorbia pulcherrima somatic embryo or Euphorbia pulcherrima plant obtained by or obtainable by a process of the invention. The invention also relates to plant material (for example, cuttings, flowers, bushes and trees) obtained from or obtainable from the aforementioned Euphorbia pulcherrima somatic embryos or Euphorbia pulcherrima plants.

In particular, the invention encompasses transgenic seeds and cuttings, and meristems by in vitro culture obtained from or obtainable from the aforementioned Euphorbia pulcherrima somatic embryos or Euphorbia pulcherrima plants which comprise the gene construct.

The invention also provides a Euphorbia pulcherrima plant, plant cell, plant tissue or plant protoplast which comprises in its genome a region of DNA having the nucleotide sequence of at least one T-DNA border.

Preferably, the T-DNA border is a right T-DNA border.

In one embodiment, the Euphorbia pulcherrima plant, plant cell, plant tissue or plant protoplast comprises in its genome a genetic construct comprising a selectable marker and reporter gene flanked by one or more T-DNA borders.

The invention also relates to plant material (for example, cuttings, flowers, bushes and trees) obtained from or obtainable from the aforementioned Euphorbia pulcherrima plants, plant cells or plant tissues

In particular, the invention encompasses transgenic seeds and cuttings, meristems by in vitro culture obtained from or obtainable from the aforementioned Euphorbia pulcherrima plants, plant cells or plant tissue which comprise the genetic construct.

### BRIEF DESCRIPTION OF FIGURES

FIGS. 1A and 1B. Poinsettia cv. Millennium showing mosaic symptoms caused by poinsettia mosaic virus (PnMV).

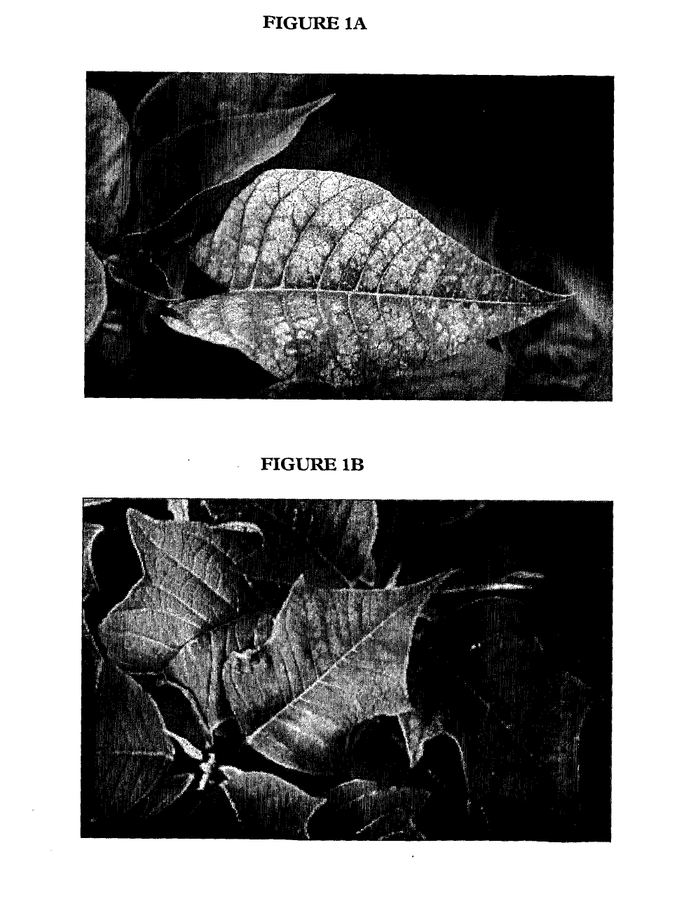

FIG. 2: Diagrammatic representation of the three hpRNA constructs.

CP: coat protein region (500 by in length).
R2 and R3: RNA dependent RNA polymerase (RdRp) regions about 500 by in length.

FIGS. 3A-C: Somatic embryogenesis derived from Agrobacterium-mediated transformation in poinsettia cv. Millennium.

FIG. 4: Somatic embryogenesis derived from Agrobacterium-mediated transformation in poinsettia cv. Millennium.

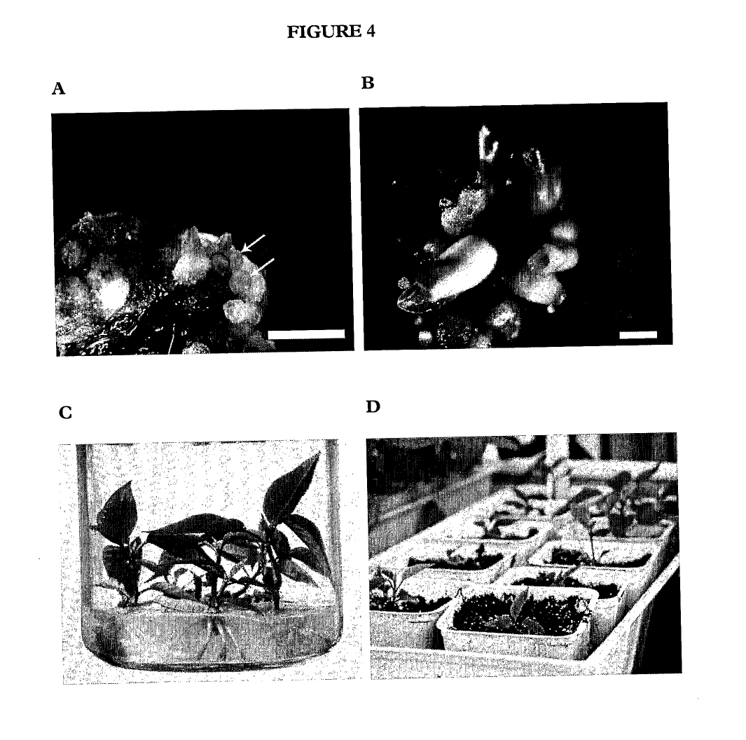

(A) Embryogenic structure and globular stage somatic embryos (arrows) appeared on the callus after four weeks of culture (bar: 1 mm).
(B) Cotyledonary stage of somatic embryos (bar: 1 mm).
(C) Plantlets deriving from somatic embryos on RIM medium.
(D) Regenerated plants established in the greenhouse.

FIG. 5: Agrobacterium-mediated transformation of poinsettia cv. Millennium. (A)

Somatic embryos induced from internode explants after transformation. (B)
Regenerated poinsettia after kanamycin selection.
(C) and (D) Transgenic poinsettia plants in the greenhouse.

FIG. 6: A poinsettia transformant growing in the greenhouse.

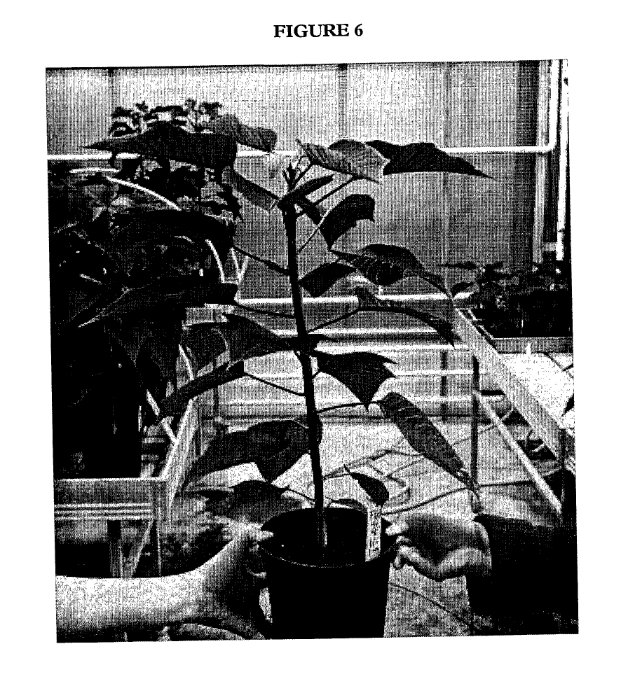

FIG. 7: PCR screening of DNA samples from transgenic plants with the vector pR3 construct. Ctrl: untransformed plants serving as control. Plasmid: positive control. Lanes form R3-1 to R3-11 are the putative transgenic plants.

FIG. 8: Southern analysis of HindIII digested genomic DNA from transgenic poinsettia and control. Ctrl: untransformed control; Plasmid: plasmid DNA as positive control; Lane 2-15: poinsettia transformants.

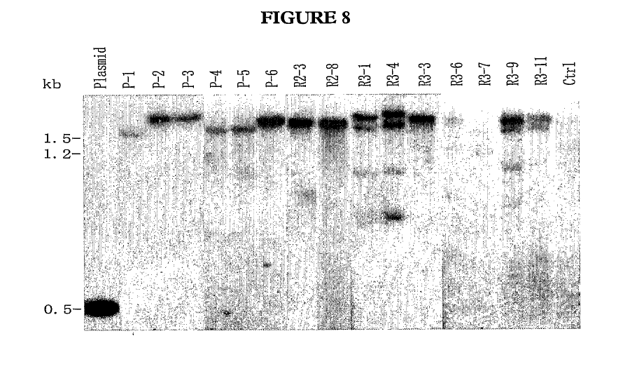

FIG. 9: Plasmid pKES18 corrected 2.

FIG. 10: PCR-positive transgenic Poinsettia cv. Cristella embryos.

### EXAMPLES

The following abbreviations are used herein:

| | |
|---|---|
| 2iP | (2-isopentenyl)adenine |
| BAP | 6-benzylaminopurine |
| CI | callus induction medium |
| CP | coat protein |
| CPA | (4-chlorophenoxy)acetic acid |
| HSFM | hormone free half strength MS medium |
| IAA | indole-3 acetic acid |
| MS | Murashige and Skoog medium (1962) |
| MS-2 | MS medium + 2% sucrose |
| NAA | 1-naphthaleneacetic acid |
| PnMV | Poinsettia mosiac virus |
| RdRp | RNA dependent RNA polymerase |
| RI | root induction medium |
| SEI | somatic embryo induction medium |
| SEM | somatic embryo maturation medium |

### Derivatives

Whilst specific reference is made herein to the above media and products, the invention also encompasses the use of derivatives of the above media/products and equivalents thereof. For example, whilst reference is made herein to 2iP, BAP, CAP, IAA and NAA, the invention also relates to derivatives (for example acid addition salts) and obvious variants which have the same or equivalent biological effect.

The present invention is further defined in the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

The disclosure of each reference set forth herein is incorporated herein by reference in its entirety.

TABLE 1
Media names and compositions

| Medium | MS | Sucrose | CPA | BAP | NAA | 2iP | IAA |
|---|---|---|---|---|---|---|---|
| CI | Full strength | 3% | 0.2 mg/l | 0.2 mg/l | — | — | — |
| SEI | Full strength | 3% | — | — | 0.3 mg/l | 0.15 mg/l | — |
| SEM | Full strength | 3% | — | 0.05 mg/l | — | — | — |
| RI | ½ strength | 2% | — | — | — | — | 2 mg/l |
| HFMS | ½ strength | 2% | — | — | — | — | — |

### Example 1

### Preparation of Plant Material

Euphorbia pulcherrima cv. Millennium plants were kindly supplied by Kristiansen nursery, Grimstad, Norway, in the year 2000. The original stock plants were subjected to heat therapy to eliminate poinsettia mosaic virus (PnMV). PnMV-free Millennium cuttings were grown in the greenhouse under a photoperiod of 16 h light and 8 h dark with a temperature of 22° C. Internode stem explants from eight to ten weeks old Millennium plants were used for Agrobacterium-mediated transformation of poinsettia.

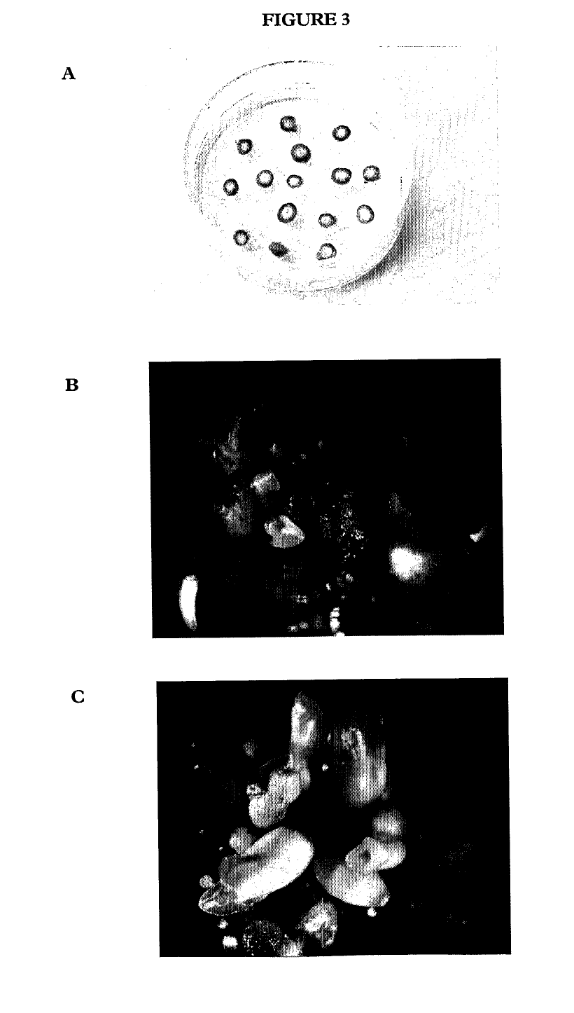

Stem explants 0.5-1.5 cm long taken below the shoot apical meristem from Millennium plants were excised and used in the present study (FIG. 3). The stem explants were sterilized with an alternative method: 70% alcohol for 1 min followed by 5 min with 1% NaOCl and 3 rinsings with sterile deionized and autoclaved water for 3, 10 and 20 min. After sterilization, stem explants were placed on callus induction (CI) medium containing 0.2 mg L⁻¹ CPA and 0.2 mg L⁻¹ BAP (Table 1) at 24° C. in dark for one day before Agrobacterium inoculation.

### Example 2

### Agrobacterium Strains and Hairpin (hp) RNA Constructs

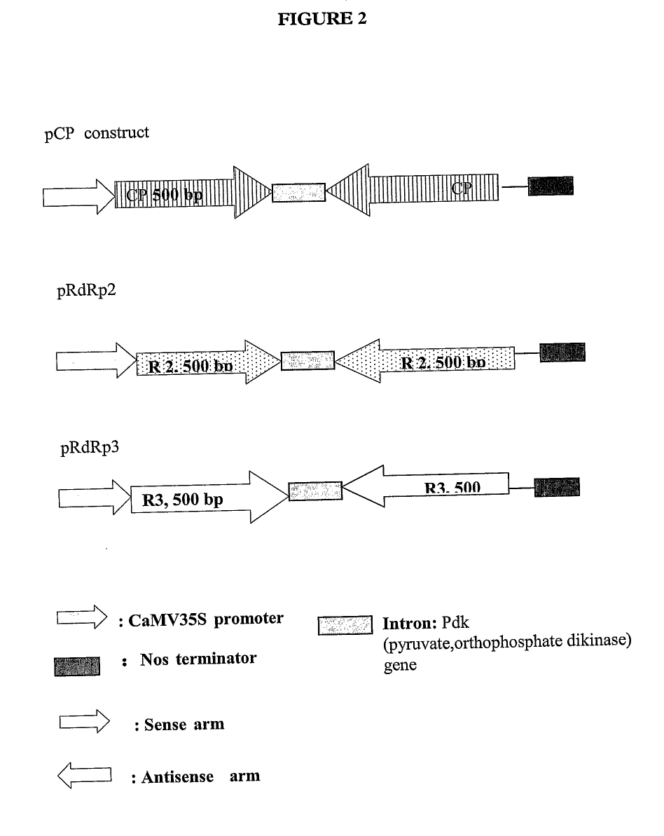

Agrobacterium tumefaciens strain LBA4404 (Hoekema et al. 1983, Nature 303:179-181); Invitrogen), harbouring three hairpin (hp) RNA constructs, namely pCP, pR2 and pR3, derivatives of the original pHANNIBAL. provided by CSIRO Plant Industry (Canberra, Australia), was used in all the experiments. The constructs were cloned into pART27, a binary plasmid at Not1 site. The diagrammatic presentations of those hpRNA constructs are shown in FIG. 2. The pCP construct contains a 500 by fragment of coat protein (CP) gene inserted in sense and antisense orientations interrupted by a intron pdk (pyruvate, orthophosphate dikinase) gene, whereas the pR2 and pR3 constructs, possessing gene fragments (entitled R2 and R3) of RNA dependent RNA polymerase (RdRp) region about 500 by in length and interrupted by the same pdk gene respectively (FIG. 2). CP, R2 and R3 fragments were amplified by PCR, cloned into E. coli before being ligated into the defined restriction sites flanking the intron pdk gene. All the three expression cassettes were under the control of the CaMV ³⁵S promoter. For selection, the neomycin phosphotransferase II (nptII) gene conferring kanamycin resistant was used under the control of nopaline synthase promoter (Nos-P).

### Example 3

### Agrobacterium-Mediated Transformation

A. tumefaciens strain LBA4404, carrying the plasmid pCP, pR2 and pR3, was grown overnight in 15 ml liquid LB medium (Sigma, USA) supplemented with 50 mg L⁻¹ kanamycin (Duchefa) at 28° C. with shaking at 200 rpm until an OD600=0.6 was reached. The bacterium suspension was pelleted at 2700 rpm for 10 min, washed with Murashige & Skoog (MS, Murashige & Skoog, 1962, A revised medium for rapid growth and bioassays with tobacco tissue cultures. Physiol. Plant 15(3):473-497) basal medium supplemented with 2% sucrose (MS-2), and resuspended in 10 ml MS-2.

The pre-prepared stem explants excised into stem segments with 1-1.5 mm thickness (FIG. 2) were infected with Agrobacterium suspension for 5 min with gentle shaking by hand, blotted briefly with sterile filter paper (Walkman), and placed on CI medium (Table 1) at 24° C. in dark for 72 h without selection. Subsequently, the explants were blotted gently on sterile filter paper and transferred to the CI medium supplemented with 400 mg L⁻¹ claforan.

### Example 4

### Somatic Embryogenesis and Regeneration of Transgenic Plants

After 7-10 days on CI medium supplemented with 400 mg L⁻¹ claforan, embryogenic calli appeared on brownish stem segment explants were transferred to somatic embryo induction (SEI) medium containing 0.3 mg L⁻¹ NAA and 0.15 mg L⁻¹ 2iP (Table 1) supplemented with 400 mg L⁻¹ claforan for somatic embryogenesis for 2 weeks. After the first round sub-culture, embryogenic calli were transferred to SEI medium supplemented with 500 mg L⁻¹ claforan and 25 mg L⁻¹ kanamycin. The globular stage of the somatic embryos became visible on the calli after 2-3 round sub-cultures, i.e. after 4-6 weeks. After further a couple of subcultures, the SEI medium was supplemented with 250 mg L⁻¹ claforan and 10 mg L⁻¹ kanamycin. The somatic embryos developed after 10 weeks were then transferred to somatic embryo maturation (SEM) medium containing 0.05 mg L⁻¹ BAP (Table 1). Plantlets derived from somatic embryos were subsequently cultured on root induction (R1) medium containing ½ strength MS and 2 mg L⁻¹ IAA or hormone free ½ strength MS (HFMS) medium (Table 1) for root induction. Plants with well-developed roots were transferred to soil and grown in the greenhouse at 22° C.

Light conditions were 23 μE m⁻² s⁻¹ for the callus and somatic embryos on CI, SEI and SEM media, whereas 30 μE m⁻² s⁻¹ for plantlets in RIM and HFMS media with a 16 h photoperiod. Light microscopy connected with a digital camera was used to follow up the development of somatic embryos.

### Example 5

### Polymerase Chain Reaction (PCR) for Screening of Transgenic Poinsettia Lines

Three primer pairs designed for amplifying the CP, R2 and R3 fragments were:

CP: forward
(SEQ ID NO: 1)
5′ acgtctagaAACCACGTCGACTCCACTCCAT 3′
CP: reverse
(SEQ ID NO: 2)
5′ agcatcgatAGCTTGCCGCTCACCAGCAC 3′
R2: forward
(SEQ ID NO: 3)
5′ acgtctagaTTTAGCAAAACGCAGCACAAAATCA 3′
R2: reverse
(SEQ ID NO: 4)
5′ agcatcgatTCTCCAGACACCATGATTGGGTG 3′
R3: forward
(SEQ ID NO: 5)
5′ acgtctagaTTCGCTTTAAAACAGAAAGCACCA 3′
R3: reverse
(SEQ ID NO: 6)
5′ agcatcgatGCCTCGTAGCTTGGTTGGGTT 3′

The HotStarTaq PCR kit purchased from Qiagen (Valencia, Calif.) and standard PCR kit from Applied Biosystems (manufactured by Roche Molecular Systems, Inc., New Jersey, USA) were utilized in PCR screening of transformants. For HotStartTaq PCR kit, 20 μl reaction mixture containing 2× HotstarTaq Mastermix, 0.4 μM of each primer, 0.1 μg template DNA and H2O was subjected to PCR amplification with following conditions: 15 min at 95° C. (1 cycle), 30 sec at 95° C., 30 sec at 55° C., 1 min at 72° C. (35 cycles) and a final extension 10 min at 72° C. (1 cycle). When the standard PCR kit was used, a 20 μl reaction mixture consisted of 10×PCR buffer, 3 mM MgCl, 250 μM each of dNTPs, 0.4 μM of each primer, and 0.1 μg template DNA and 1 U Taq DNA polymerase. The amplification was performed in Applied Biosystems 96 Thermal Cycler (Applied Biosystems) with program: 5 min at 95° C. (1 cycle), 30 sec at 95° C., 30 sec at 56° C., and 1 min at 72° C. (35 cycles), and a final elongation 7 min at 72° C. (1 cycle). The PCR fragments produced were separated on a 0.8% (W/V) agarose gel and evaluated.

### Example 6

### Southern Blot Analysis of Transformants

For Southern blot analysis, total genomic DNA was isolated from young leaves of control and transformed plants following a modified CTAB protocol as described by Rogers S O, Bendich A J (1988) (Extraction of DNA from plant tissues. In: Gelvin S B, Schilperoort R A (eds) Plant Molecular Biology Manual, pp A6:1-10. Boston, Mass.: Kluwer. Academic Publishers). Ten micrograms of genomic DNA was digested with the restriction enzymes HindIII and EcoRI respectively for 4 h and separated on a 1% (WN) TBE agarose gel overnight at 37 V. After transfer to a nylon membrane, the blot was prehybridized and hybridized with ³²P labelled probe of 1.5 kb in size consisted of the three 500 by fragments, i.e. CP, R2 and R3. Southern blot hybridization was performed and membranes were washed and exposed to X-ray films basically as described by Sambrook et al. (1989) (Molecular cloning: A laboratory manual, 2nd ed., Cold Spring Harbor Laboratory Press, Plainview, N.Y.).

### Example 7

### Resistance Assay after Inoculation with Poinsettia Mosaic Virus (PnMV)

Transgenic poinsettia lines were multiplied vegetatively using cuttings. The rooted cuttings were inoculated by sap inoculation at 3-4 leaves stage. The inoculum consisted of a mixture of PnMV multiplied in Nicotiana benthamiana and in Poinsettia pulcherrima diluted about 1:5 w/v. The inoculated plants were tested for PnMV several times by ELISA during the 4-6 months observation period.

### Example 8

### Elisa Assays

Enzyme-linked immunosobent assay, ELISA, was performed with antisera from DSMZ (Deutsche Sammlung von Mikroorganismen and Zellkulturen GmbH, Braunschewig, Germany), according to the manufacturer's instructions.

After inoculation with virus, leaf samples from both inoculated and new leaves were harvested several times during the following growth and observation period.

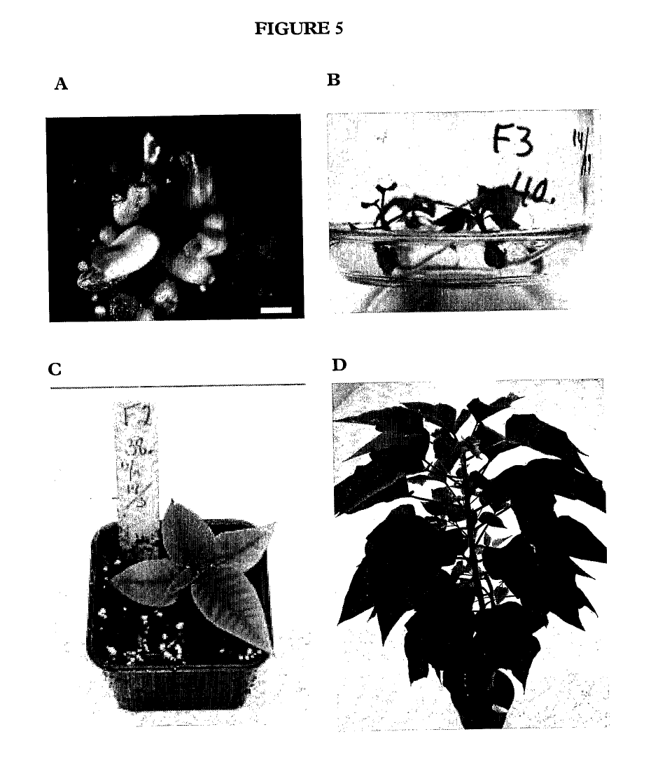

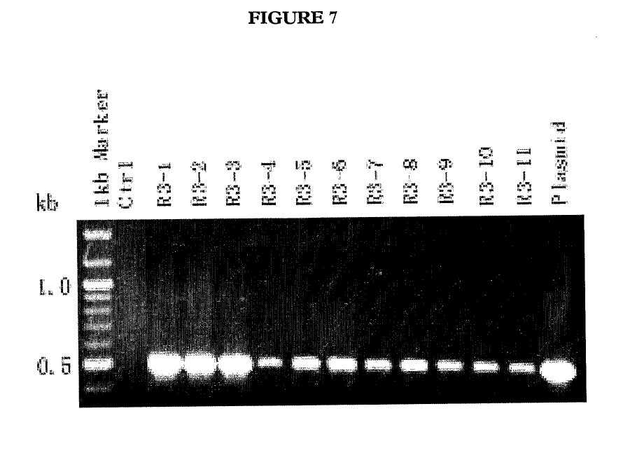

Based on PCR and Southern blot analyses, transgenic poinsettia lines by Agrobacterium-mediated transformation were obtained (FIGS. 5a and 5b). Stable integrations of transgenes were confirmed by Southern blot analysis (FIG. 7).

Inoculation experiment with PnMV followed by Elisa assays revealed PnMV resistance in transgenic poinsettia lines.

### Example 9

### Transformation of Poinsettia cv. “Cristella”

The Agrobacterium-mediated transformation of Euphorbia pulcherrima cv. Cristella was performed based on our protocol (Clarke et al. 2008, Plant Cell Rep. 27:1027-1038, the contents of which are incorporated in entirety).

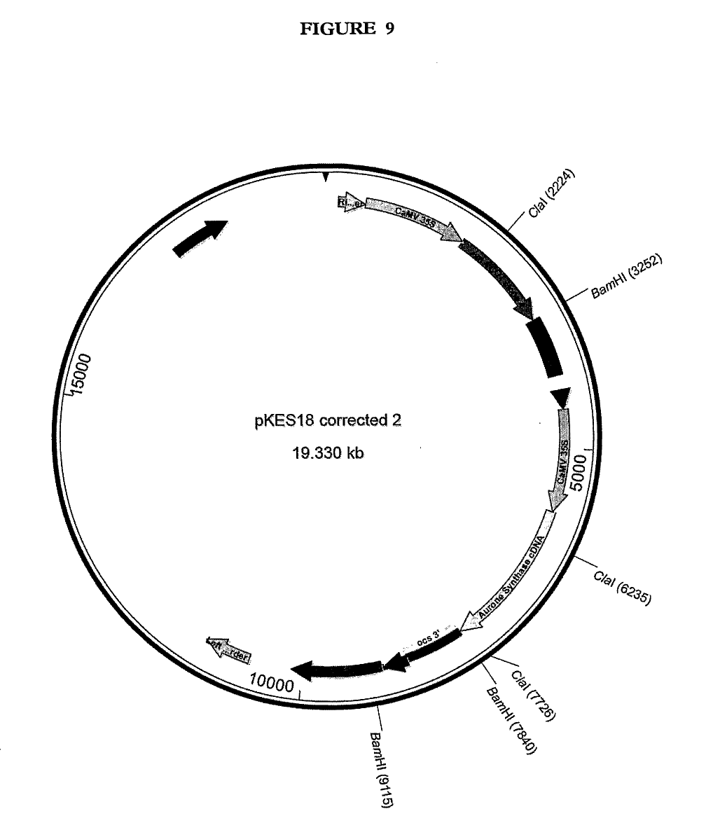

A. tumefaciens strain LBA4404 was used, harbouring the plasmid pKES18 (FIG. 9) which contains the A. majus aureusidin synthase (AmAS1) and chalcone 4′-O-glucosyltransferase (4′CGT) genes for production of yellow-colored poinsettia (Ono et al. 2006, PNAS vol. 103, no. 29: 11075-11080, the contents of which are incorporated in entirety).

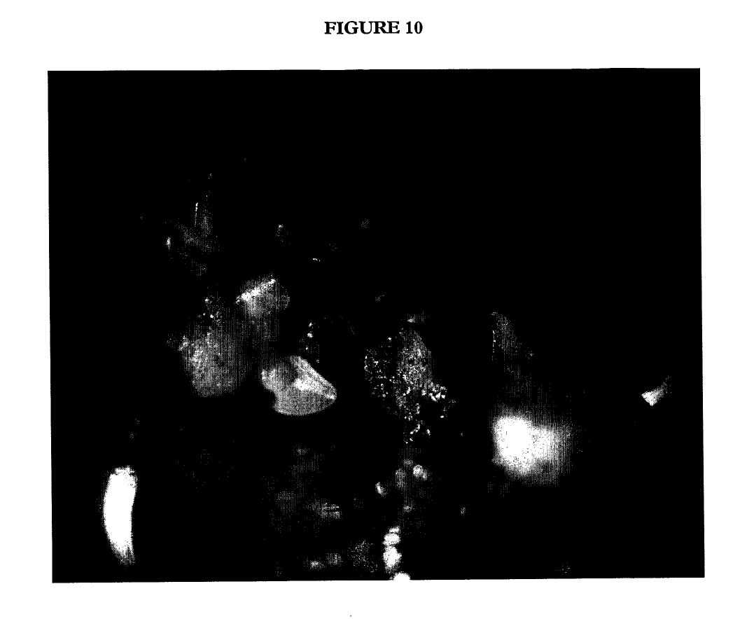

Internode stem explants of Cristella taken from 8-10 week old poinsettia plants derived from cuttings were disinfected as described in Example 1, excised into stem segments with 1-1.5 mm thickness and inoculated with Agrobacterium suspension for 5 min with gentle shaking. All the procedures including infection, co-cultivation, selection and regeneration were identical to that for Millennium as described above. PCR-positive transgenic Cristella embryos are shown in FIG. 10.

### Sequence Listing Free Text

### SEQ ID NO: 1

<223> Forward PCR primer used to amplify CP fragment

### SEQ ID NO: 2

<223> Reverse PCR primer used to amplify CP fragment

### SEQ ID NO: 3

<223> Forward PCR primer used to amplify R2 fragment

### SEQ ID NO: 4

<223> Reverse PCR primer used to amplify R2 fragment

### SEQ ID NO: 5

<223> Forward PCR primer used to amplify R3 fragment

### SEQ ID NO: 6

<223> Reverse PCR primer used to amplify R3 fragment

1. A process for producing a transgenic Euphorbia puleherrima somatic embryo, comprising the steps of:
(a) culturing a transformable Euphorbia pulcherrima plant material, wherein the plant material is a plant cell, plant tissue, explant or protoplast, on or in a callus-inducing (CI) medium comprising (4-chlorophenoxy)acetic acid (CPA) and 6-benzylaminopurine (BAP);
(b) inoculating the transformable Euphorbia pulcherrima plant material with Agrobacterium which contains a gene construct comprising a selectable marker gene and a reporter gene and which is capable of transferring the gene construct to the plant material;
(c) culturing the transformable Euphorbia pulcherrima plant material after inoculation with Agrobacterium on or in a CI medium comprising CPA and BAP in the absence of the selection agent and an anti-Agrobacterium agent;
(d) culturing the transformable Euphorbia pulcherrima plant material on or in a CI medium comprising CPA and BAP in the presence of an anti-Agrobacterium agent and the selection agent;
(e) culturing the embryogenic calli thus produced on or in a somatic embryo induction medium comprising 1-naphthaleneacetic acid (NAA) and (2-isopentenyl)adenine (2iP) in the presence of an anti-Agrobacterium agent and the selection agent; and
(f) culturing the somatic embryos thus produced on or in a somatic embryo induction medium comprising NAA and 2iP in the presence of an anti-Agrobacterium agent and the selection agent, and optionally subculturing the somatic embryos one or more additional times.

2. A process as claimed in claim 1, which additionally comprises the step:
(g) culturing the somatic embryos on or in a somatic embryo maturation medium comprising BAP.

3. A process as claimed in claim 2, which additionally comprises the step:
(h) culturing plantlets derived from the somatic embryos on or in root induction medium.

4. A process as claimed in claim 1, wherein the Euphorbia pulcherrima is Euphorbia pulcherrima Willd. Ex Klotsch (poinsettia).

5. A process as claimed in claim 4, wherein the Euphorbia pulcherrima cultivar is Millennium, Cristella, Angelika, Lilo or Cortez.

6. A process as claimed in claim 1, wherein the plant material is a plant cell, callus, immature or somatic embryo, shoot apical meristem, leaf explant, stem explant or protoplast.

7. A process as claimed in claim 6, wherein the plant material is a stem explant.

8. A process as claimed in claim 1, wherein the plant from which the plant material has been obtained is 6-8 weeks old.

9. A process as claimed in claim 1, wherein in Step (a), the CI media comprises 0.1-0.35 mg L⁻¹ CPA.

10. A process as claimed in claim 1, wherein in Step (a), the CI media comprises 0.1-0.35 mg L⁻¹ BAP.

11. A process as claimed in claim 1, wherein in Step (a), the plant material is cultured on or in callus inducing (CI) media comprising CPA and BAP for 20-28 hours.

12. A process as claimed in claim 1, wherein in Step (a), the plant material is cultured on or in CI medium comprising CPA and BAP in the dark or under reduced light conditions.

13. A process as claimed in claim 1, wherein the Agrobacterium is Agrobacterium tumefaciens.

14. A process as claimed in claim 13, wherein the Agrobacterium is Agrobacterium tumefaciens strain LBA 4404.

15. A process as claimed in claim 1, wherein the gene construct is a DNA plasmid or vector which comprises a selectable marker gene and a reporter gene flanked by at least one T-DNA border.

16. A process as claimed in claim 1, wherein the gene construct comprises:
(i) a left Agrobacterium T-DNA border sequence;
(ii) a selectable marker gene comprising:
a promoter which functions in Euphorbia pulcherrima plant cells
a nucleotide sequence encoding a selectable marker polypeptide
a 3′ non-translated region encoding a terminator sequence
(iii) a reporter gene comprising:
a promoter which functions in Euphorbia pulcherrima plant cells
a nucleotide sequence of interest
a 3′ non-translated region encoding a terminator sequence
(iv) a right Agrobacterium T-DNA border sequence.

17. A process as claimed in claim 1, wherein the selectable marker gene encodes a polypeptide which confers resistance to the selection agent.

18. A process as claimed in claim 1, wherein the selection agent is an antibiotic or a herbicide.

19. A process as claimed in claim 18, wherein the selectable marker gene is neomycin phosphotransferase II (nptII) and the selection agent is kanamycin.

20. A process as claimed in claim 1, wherein the reporter gene comprises:
a disease resistance gene or pathogenesis related (PR) protein-encoding gene;
a gene which encodes a polypeptide which modifies a known phenotypic trait or introduces a new phenotypic trait to the plant;
a gene which encodes a polypeptide which modifies the colour of all or part of the plant or affects the ornamental value of the plant; or
a gene-silencing construct.

21. A process as claimed in claim 1, wherein the reporter gene comprises a nucleic acid molecule which encodes a fragment of the Poinsettia Mosaic Virus (PnMV) coat protein.

22. A process as claimed in claim 1, wherein the reporter gene comprises a nucleic acid molecule which encodes a fragment of an RNA dependent RNA polymerase.

23. A process as claimed in claim 1, wherein prior to Step (b), the Agrobacteria are grown to an appropriate OD₆₀₀=0.5-1.0.

24. A process as claimed in claim 1, wherein in Step (b), the Agrobacterium are co-cultivated with the plant material for 2-20 minutes.

25. A process as claimed in claim 1, wherein in Step (c), the Agrobacterium are co-cultured with the plant material at a temperature of 20-28° C.

26. A process as claimed in claim 1, wherein in Step (c), the Agrobacterium are co-cultured with the plant material for 24-120 hours.

27. A process as claimed in claim 1, wherein in Step (c), the Agrobacterium are co-cultured with the plant material in the dark or under reduced light conditions.

28. A process as claimed in claim 1, wherein in Step (d), the concentration of the anti-Agrobacterium agent is 300-500 mg L⁻¹.

29. A process as claimed in claim 1, wherein in Step (d), the anti-Agrobacterium agent is claforan.

30. A process as claimed in claim 1, wherein in Step (d), the time that the plant material is cultured on or in the CI medium is within the range of 2-15 days.

31. A process as claimed in claim 1, wherein in Step (d), the light conditions are 20-26 μE m⁻² s⁻¹.

32. A process as claimed in claim 1, wherein in Step (d), the photoperiod is 14-18 hours.

33. A process as claimed in claim 1, wherein in Step (e), the somatic embryo inducing media comprises 0.1-0.4 mg L⁻¹ NAA.

34. A process as claimed in claim 1, wherein in Step (e), the somatic embryo inducing media comprises 0.10-0.30 mg L⁻¹ 2iP.

35. A process as claimed in claim 1, wherein in Step (e), the concentration of the anti-Agrobacterium agent is 300-500 mg L⁻¹.

36. A process as claimed in claim 1, wherein in Step (e), the anti-Agrobacterium agent is claforan.

37. A process as claimed in claim 1, wherein in Step (e), the time that the embryogenic calli are cultured on or in the somatic embryo induction media is 10-18 days.

38. A process as claimed in claim 1, wherein in Step (e), the light conditions are 20-26 μE m⁻² s⁻¹.

39. A process as claimed in claim 1, wherein in Step (e), the photoperiod is 14-18 hours.

40. A process as claimed in claim 1, wherein the selection marker gene is nptII.

41. A process as claimed in claim 1, wherein the selection agent is kanamycin.

42. A process as claimed in claim 1, wherein in Step (e), the concentration of selection agent used is 10-40 mg L⁻¹.

43. A process as claimed in claim 1, wherein in Step (f), the concentration of anti-Agrobacterium agent used is 300-700 mg L⁻¹.

44. A process as claimed in claim 1, wherein in Step (f), the anti-Agrobacterium agent is claforan.

45. A process as claimed in claim 1, wherein in Step (f), the somatic embryos are optionally subcultured a further 2-4 times.

46. (canceled)

47. A Euphorbia pulcherrima somatic embryo or Euphorbia pulcherrima plant obtained by or obtainable by a process as claimed in claim 1.

48. Seeds or plant material obtained from or obtainable from the somatic embryo or plant as claimed in claim 47 and which comprises in its genome a region of DNA having the nucleotide sequence of at least one T-DNA border.

49. Seeds or plant material obtained from or obtainable from the somatic embryo or plant as claimed in claim 48 and which comprises in its genome a genetic construct comprising a selectable marker and reporter gene flanked by one or more T-DNA borders.

50. Euphorbia pulcherrima seeds or plant material which comprises in its genome a genetic construct comprising a selectable marker and a reporter gene flanked by one or more T-DNA borders.

51. Euphorbia pulcherrima seeds or plant material as claimed in claim 50, wherein the gene construct comprises:
(i) a left Agrobacterium T-DNA border sequence;
(ii) a selectable marker gene comprising:
a promoter which functions in Euphorbia pulcherrima plant cells
a nucleotide sequence encoding a selectable marker polypeptide
a 3′ non-translated region encoding a terminator sequence
(iii) a reporter gene comprising:
a promoter which functions in Euphorbia pulcherrima plant cells
a nucleotide sequence of interest
a 3′ non-translated region encoding a terminator sequence
(iv) a right Agrobacterium T-DNA border sequence.
